(19) United States
(12) Patent Application Publication
(10) Pub. No.: US 2011/0021796 A1
(43) Pub. Date: Jan. 27, 2011

(54) LIPID PURIFICATION PROCESS

(75) Inventor: Asgeir Saebo, Sovik (NO)

Correspondence Address: Ballard Spahr LLP, SUITE 1000, 999 PEACHTREE STREET, ATLANTA, GA, 30309-3915, US

(73) Assignee: PHARMA MARINE AS a foreign corporation

(21) Appl. No.: 12/812,515

(22) PCT Filed: Jan. 19, 2009
(86) PCT No.: PCT/GB2009/000141

§ 371 Date: Oct. 6, 2010

(30) Foreign Application Priority Data
Jan. 17, 2008 (GB) 0800875.7

(51) Int. Cl. C11B 3/10; C07C 53/00; B01D 15/08

(52) U.S. Cl. 554/193; 554/1; 554/227; 210/269

(57) ABSTRACT

A process for purification of a liquid lipid from a bed of a particulate absorbent, where a supercritical fluid is passed through said bed to release lipids retained on the absorbent. Also apparatus for use in the performance of the process, lipids purified using the process and by-products of the process. The process enables enhanced removal of retained lipids from the particulate absorbent, e.g. removal of plant oils from bleaching clays.

This invention relates to a process for purification of a liquid lipid, to apparatus for use in the performance of the process, to the purified lipid, and to by-products of the process.

In the purification of many lipids (e.g. oils or fats extracted from life forms, such as plant, animal or marine life forms) it is common to contact the lipid in liquid form with a particulate absorbent, for example bleaching clay, to remove undesired impurities, for example chlorophylls or carotenoids. Generally this is done on a batch basis, by mixing the absorbent and the lipid and later draining off the purified lipid.

The remaining absorbent however can retain a very high lipid content, e.g. 30-40 wt. % when bleaching clays are used to treat plant oils, and this is problematic to dispose of and represents a wastage of the retained lipid. While disposal of spent bleaching clay in landfills has been a standard technique, this is environmentally unfriendly and may even pose a threat of spontaneous combustion. The scale of the problem is huge—it has been estimated that 600,000 tons of spent bleaching clay is produced annually, about 70,000 tons in Malaysia alone (see Ng et al JAOCS 74: 963-969 (1997)).

There have been proposals to subject the spent bleaching clay to solvent extraction to retrieve much of the retained lipid, but the treated clay still has to be disposed of.

We have now found that these problems may be addressed by cycling between lipid purification and absorbent regeneration using a bed of particulate absorbent through which the lipid to be purified is passed and through which a supercritical fluid is then passed to regenerate the absorbent by extracting retained lipids therefrom.

Thus viewed from one aspect the invention provides a process for the purification of a liquid lipid wherein in one process stage a liquid lipid is passed through a bed of a particulate absorbent in a vessel and purified lipid is removed from the vessel and in a further process stage a supercritical fluid is passed through said bed to release lipids retained on the absorbent and the released lipids are removed from the vessel. These two process stages may be alternated between repeatedly and the lipid may be passed through the absorbent bed repeatedly until the desired degree of purification is achieved.

Viewed from a further aspect the invention provides apparatus for the purification of a liquid lipid comprising a vessel containing a bed of particulate absorbent and having a first inlet port for admitting liquid lipid into said vessel, a first outlet port for removing purified liquid lipid from said vessel, a second inlet port for admitting a supercritical fluid into said vessel, a second outlet port for removing from said vessel lipids released from said absorbent by said supercritical fluid, said apparatus further comprising a source of supercritical fluid connected by a first conduit to said second inlet port and a condenser connected by a second conduit to said second outlet port.

Viewed from a still further aspect the invention provides a purified lipid product obtainable by (or obtained by) the process of the invention, if desired followed by further formulation steps. Such formulation steps may for example comprise: admixture with further materials; emulsification, encapsulation, tableting and the like; cooking; or a combination of such steps.

Viewed from a still further aspect the invention provides a by-product, e.g. a lipid, released by supercritical fluid in the process of the invention.

The liquid lipid purified according to the process of the invention may be any lipid mixture which is liquid at ambient temperature or which may be melted to be purified in liquid form. Typically the lipid will be introduced into the vessel in liquid form at a temperature of 10 to 150° C., especially 20 to 120° C., particularly 50 to 110° C. The lipid is preferably an oil or fat of biological origin, e.g. from a mammal, a plant or a marine organism, for example a multi-cellular marine life form such as fish. The lipid is especially preferably a plant or marine oil.

By purification of the lipid is meant partial or total removal of an undesired component present in the lipid. In the case of processing of oils and fats for production of edible lipids, purification may take the form of a bleaching and/or deodorizing process and for this reason the lipid is preferably introduced into the vessel at elevated temperature, e.g. as described in U.S. Pat. No. 4,230,630.

The lipid may be introduced into the vessel at ambient pressure and allowed to percolate through the absorbent bed. However it will generally be desirable to apply a pressure differential to force the lipid through the absorbent bed. The pressure applied may for example be up to 1500 bar, but will preferably be 50 to 600 bar, especially 90 to 160 bar. Typically the lipid will be applied to the top of the bed and withdrawn from the bottom of the bed. In one embodiment, a multi-tiered bed may be used and lipid may be applied to the top of one or more of the tiers and withdrawn from the base of one or more tiers. In this way, by monitoring the purity of the lipid being withdrawn, it is possible to gradually change the set of tiers through which the lipid passes so as to optimize the pressure differential required to pass the lipid through sufficient unexhausted absorbent to achieve the desired degree of purification. Thus for example with fresh absorbent a lower pressure differential can be used to pass the lipid through fewer tiers of absorbent.

The absorbent may be any particulate absorbent material capable of absorbing the contaminants to be removed from the lipid. Preferably the absorbent is a clay (e.g. Fuller's earth, soap clay, acid-activated montmorillonite, attapulgite, volcanic clay or a synthetic clay). However, any of the particulate absorbents used for lipid purification, in particular plant and marine oil purification, may be used. Other materials that can be used include silica, alumina, molecular sieves, macroreticulate polymers, carbon, aluminosilicates, etc.

Particularly desirably, the absorbent comprises a smectite clay, e.g. a bentonite.

The particle size of the absorbent may typically be in the micrometer to millimetre range. The depth of the absorbent bed will be dependent on the nature and particle size of the absorbent and the degree to which purification is required. Bed depth however will typically be 10 to 10000 cm, especially 30 to 400 cm. Bed cross-sectional above will of course depend upon the mass throughput required of the purification plant, but typically may be 30 to 150 cm².

The absorbent may be a single material, e.g. a clay, or a combination of materials, e.g. a clay and carbon or silica. By using a combination of materials, removal of desirable components from the supercritical fluid may be enhanced. In a particularly preferred embodiment, a plurality of absorbent-containing vessels are arranged in series containing different, or differently mixed, absorbents so as to optimize the capture of the desirable components.

If desired, the absorbent may be disposed in the absorbent bed in a channelized fashion.

The supercritical fluid used for absorbent regeneration may be any material which in supercritical state acts as a solvent for the lipid. Typically it will be an optionally halogenated alkane, alkene or aralkane, or an ether, water, ammonia or carbon dioxide, e.g. ethane, propane, n-butane, n-pentane, ethene, propene, cyclohexene, toluene, diethyl ether, trifluoromethane, chlorotrifluoromethane, trichlorofluoromethane, diethyl ether, etc. The use of carbon dioxide however is preferred. The temperature and pressure at which the supercritical fluid is applied to the vessel will of course depend on the location of the critical point of the material used. For carbon dioxide for example, temperature will typically be 60 to 80° C. and pressure 300 to 800 bar, e.g. 60° C. and 500 bar.

The supercritical fluid may be introduced above or below the absorbent bed but preferably is introduced below and removed from above the bed.

The supercritical fluid leaving the vessel may be freed of entrained lipid in a condenser, typically a vessel in which pressure is reduced while temperature is maintained elevated. In a preferred embodiment, two or more, preferably 2 to 4, such condensers are present in series such that successive pressure reductions and/or temperature changes in successive condensers produces different lipid condensates. For plant and marine oils for example a first condenser might bring pressure and temperature to 150 bar and 60° C. to collect free fatty acids, while a second condenser might bring pressure and temperature to 60 bar and 60° C. to collect neutral lipids.

Desirably, the supercritical fluid material is recycled into the vessel and thus following the condensers it may be fed to a holding tank. Such a tank may desirably be cooled so as to condense out any water. From the holding tank it may be fed to a compressor to reach the desired temperature and pressure for insertion back into the vessel.

The apparatus will desirably be provided with a reservoir of the material from which the supercritical fluid is to be formed, e.g. a liquid or pressurized gas reservoir.

This separation of the materials removed from the absorbent is particularly beneficial as the different materials have different beneficial end uses. The process operated in this manner is thus much more than simply the recovery of the 30-40% oil retained by spent bleaching earth before groundfill disposal as has been proposed earlier (see Ng et al supra).

In an especially preferred embodiment, following a first phase of absorbent regeneration using supercritical fluid, a second phase is effected in which a polar solvent (e.g. water or an alcohol such as C₁₋₅ alkanol, for example methanol, ethanol, n-propanol, i-propanol, n-butanol, etc.) is injected into the supercritical fluid. The use of ethanol in this regard is preferred.

In this way downstream condensers may be used to remove polar materials entrained by supercritical fluid (and of course to remove the polar solvent, e.g. for recycling). Examples of polar materials recovered include phospholipids. Where these condense out with the polar solvent, the solvent may be removed, e.g. by vacuum distillation, and returned to a polar solvent reservoir for reuse. Typically the polar solvent will be added as 5 to 20% wt/wt of the supercritical fluid. Addition may be as a liquid where the polar solvent is liquid at the temperature of the supercritical fluid, e.g. at from ambient pressure up to the pressure of the supercritical fluid.

Following the combined use of the supercritical fluid and the polar solvent, it is preferred that the particulate absorbent bed be flushed with a gas to remove retained polar solvent. The gas used for this flushing step is preferably a non-oxidising gas, e.g. nitrogen, or more preferably carbon dioxide. Any polar solvent removed by this flushing step can of course be condensed in condensers and recycled.

Before first use, the absorbent in the reactor is preferably degassed to remove oxidant gases, e.g. under vacuum and/or by flushing with a non-oxidizing gas, e.g. nitrogen or carbon dioxide. This is particularly desirable as many of the useful materials which could be retained by the absorbent are oxidizable, e.g. vitamins, and the surface area of the absorbent is large. In this way the value of the material recovered by absorbent regeneration is further increased. Particularly preferably, the process is performed without admitting air (or any other oxidising gas) into the vessel. In this way, oxidation of lipid components is avoided and the purified product will thus differ from conventionally purified lipids and more useful by-products, e.g. vitamins, can be recovered from the absorbent. The recovery of vitamin A in this fashion is an especially preferred feature of the invention.

As a later phase of the regeneration procedure, the absorbent may also be flushed with a dilute mineral acid whereby to restore its absorbency. In this way the lifetime of the absorbent before final disposal may be extended.

Desirably, the supercritical fluid supply and the condensers are connected to a plurality of absorbent containing vessels so that lipid purification may continue in one or more vessel while absorbent regeneration progresses in one or more other of the vessels.

The materials from which the vessel and the conduits, condensers and reservoirs are constructed should of course be capable of withstanding the temperatures, pressures and chemicals encountered during the process. Material selection in this regard however represents routine chemical engineering.

Illustrative embodiments of the process and apparatus of the invention will now be described with reference to the accompanying drawing, in which:

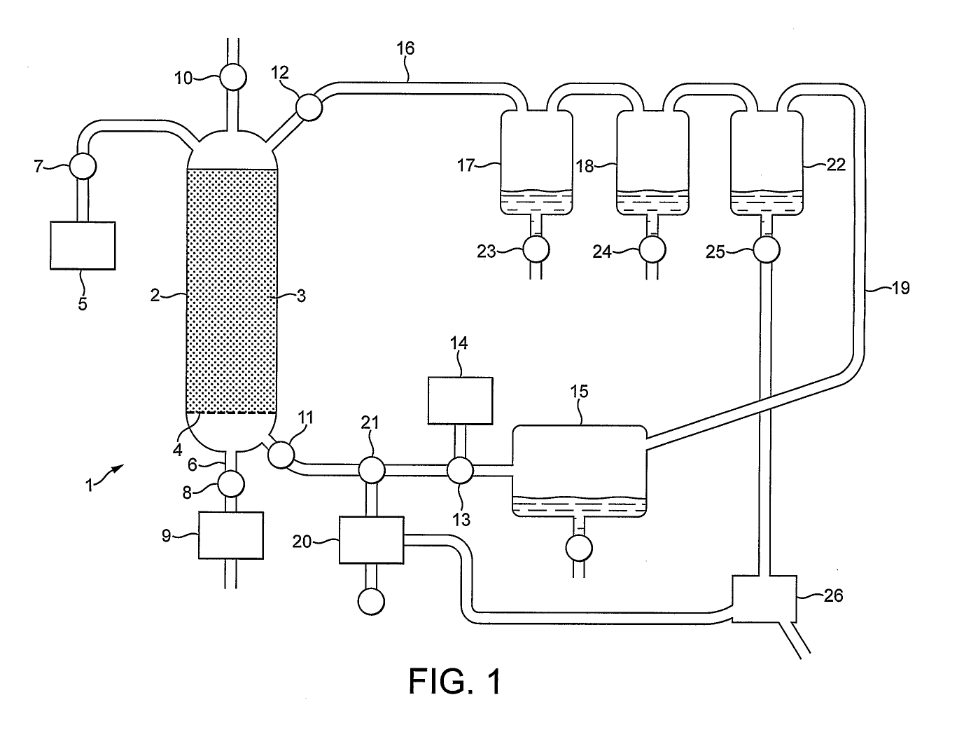

FIG. 1 is a schematic diagram of apparatus for performing the process of the invention.

Referring to FIG. 1 there is shown an apparatus 1 for plant oil bleaching. Pressure vessel 2 contains a bed 3 of bleaching clay supported on a perforated plate 4.

Plant oil is pumped from reservoir 5 into the top of vessel 2, allowed to pass through bed 3, and retrieved through outlet 6. Valves 7 and 8 are provided to halt plant oil inlet and removal. Removed plant oil is monitored for colour by online spectrometer 9.

When the detailed colour value is approaching the maximum desired, valve 7 is closed and valve 10 is opened to allow pressurized carbon dioxide to drain out oil still in the absorbent. Valves 8 and 10 are then shut and valves 11 and 12 are opened to allow supercritical carbon dioxide from compressor 13 to flow through the absorbent bed. The carbon dioxide feed to the compressor is from reservoirs 14 and/or 15. Supercritical carbon dioxide passing through valve 12 in outlet conduit 16 passes into a first condenser 17 where free fatty acids condense out through pressure drop and thence to a second condenser 18 where triglycerides condense out through pressure drop and thence via conduit 19 to carbon dioxide reservoir 15 where water is condensed out.

After the rate of liquid collection in condensers 17 and 18 reduces, ethanol is fed from reservoir 20 into the supercritical carbon dioxide stream via valve 21. Ethanol and polar materials condense out of the carbon dioxide flow in third condenser 22. The materials condensed in the condensers may be withdrawn via valves 23, 24 and 25, in the latter case passing the liquid to a distillator 26 where the ethanol is removed and returned to the ethanol reservoir.

Following absorbent bed regeneration in this fashion, valves 11 and 12 are closed and plant oil flow through the bed is resumed.

By collecting the by-products in separate condensers, the triglyceride fraction may be added to the purified plant oil.

1. A process for the purification of a liquid lipid wherein in one process stage a liquid lipid is passed through a bed of a particulate absorbent in a vessel and purified lipid is removed from the vessel and in a further process stage a supercritical fluid is passed through said bed to release lipids retained on the absorbent and the released lipids are removed from the vessel.

2. The process of claim 1, wherein the two process stages are alternated between repeatedly.

3. The process of claim 1, wherein the liquid lipid is introduced into the vessel in liquid form at a temperature of 10 to 150° C.

4. The process of claim 1, wherein a pressure differential of 50 to 600 bar is applied to force the lipid through the absorbent bed.

5. The process of claim 1, wherein the lipid is an oil or fat of biological origin, preferably a plant or marine oil

6. The process of claim 1, wherein the absorbent comprises a clay, silica, alumina, a molecular sieve, a macroreticulate polymer, carbon or an aluminosilicates, preferably the smectite clay bentonite.

7. The process of claim 1, wherein the absorbent comprises a combination of materials, preferably a clay and carbon or silica.

8. The process of claim 1, wherein the supercritical fluid comprises an optionally halogenated alkane, alkene or aralkane, or an ether, water, ammonia or carbon dioxide, preferably carbon dioxide.

9. The process of claim 1, wherein following the first phase of absorbent regeneration using supercritical fluid, a second phase is effected in which a polar solvent, preferably ethanol, is injected into the supercritical fluid and polar materials entrained by the supercritical fluid are removed.

10. The process of claim 9, wherein following the second phase the particulate absorbent bed is flushed with a gas, preferably nitrogen or carbon dioxide, to remove retained polar solvent.

11. Apparatus for the purification of a liquid lipid comprising a vessel containing a bed of particulate absorbent and having a first inlet port for admitting liquid lipid into said vessel, a first outlet port for removing purified liquid lipid from said vessel, a second inlet port for admitting a supercritical fluid into said vessel, a second outlet port for removing from said vessel lipids released from said absorbent by said supercritical fluid, said apparatus further comprising a source of supercritical fluid connected by a first conduit to said second inlet port and a condenser connected by a second conduit to said second outlet port.

12. A purified lipid product obtainable by (or obtained by) a process according to claim 1, optionally followed by one or more further formulation steps.

13. A by-product released by supercritical fluid in a process according to claim 1.
